(19) United States
(12) Patent Application Publication
(10) Pub. No.: US 2022/0125643 A1
(43) Pub. Date: Apr. 28, 2022

(54) Hygiene Pad

(71) Applicant: Demond Crump, McDonough, GA (US)

(72) Inventor: Demond Crump, McDonough, GA (US)

(21) Appl. No.: 17/078,641

(22) Filed: Oct. 23, 2020

(51) Int. Cl. A61F 13/00; A61F 13/511; A61F 13/56; A61L 15/18; A61L 15/46; A61F 13/472; A61F 13/514; A61F 13/15

(52) U.S. Cl. CPC A61F 13/00063; A61F 13/51121; A61F 13/5611; A61L 15/18; A61F 2013/15943; A61L 15/46; A61F 13/472; A61F 13/51401; A61F 13/5616

(57) ABSTRACT

The present invention is a multi-layer hygiene pad infused with graphene.

### BACKGROUND OF THE INVENTION

A World Health Organization report indicates that within two hours of a woman putting on traditional pads, about 107 different germs per cubic centimeter are developed on the surface area of the pad. When pressure is applied on the pad through sitting, because paper is not absorbent enough, the pad allows a “back flow” of the blood into the woman's reproductive system. Along with all the germs that may have developed and the chemicals in the pads, this could possibly lead to an infection in the woman's reproductive system. Not knowing what to do nor what may be happening, the woman simply takes this as normal menstrual discomfort.

Studies have revealed that menstrual sanitary pads on the market are often produced from recycled paper materials that contain print ink and other chemicals which are later bleached with harmful chemicals like chlorine.

Pads and tampons with odor neutralizers are virtually a chemical soup. They are laced with artificial colors, polyester, adhesives and other contaminants linked to, inter alia: hormone disruption, cancer, birth defects and infertility.

Most tampons aren't pure and when the chemicals come in contact with your skin they are absorbed straight into the bloodstream, putting a user at risk of Toxic Shock Syndrome (TSS), a life-threatening illness.

There is a great need for advancement in person hygiene pads.

The present invention addresses this issue.

### SUMMARY OF THE INVENTION

Although hygiene pads are typically understood and used by woman, the present invention is a hygiene pad and contemplated that men requiring such a pad, for conditions including, but not limited to, incontinence, can utilize the present invention.

In one embodiment, hygiene pads of the present invention have a soft cotton design, with a 3-dimensional layer, and leak guard sides for extra protection.

In one embodiment, hygiene pads of the present are infused with graphene. The hygiene pads provide health benefits to potentially help with circulatory health, cell activity, and metabolic health. In addition, hygiene pads of the present inhibit harmful bacteria growth.

In one embodiment, hygiene pads of the present are configured with an air-laid blockade for extra protection against undesired leakage. The innovative configuration is adept at moving the moisture away from the body.

In one embodiment, hygiene pads of the present are configured for super absorbency, absorbing up to 10 times more menstrual flow than traditional sanitary napkins.

In one embodiment, hygiene pads of the present are configured with a bottom layer having extreme breathability. This eliminates heat and undesirable moisture. The perforated bottom layer offers air dispersion to keep the area cool and most of all dry, which may also help prevent growth of harmful bacteria. The breathable bottom layer also provides a durable adhesive without the use of toxic chemicals that are detrimental to a female's most precious area, as well as the planet.

In one embodiment the invention is a hygiene pad comprising:
a proximal non-woven top sheet;
a graphene layer distal to said nonwoven top sheet;
a first air laid paper layer distal to said graphene layer;
a polymer layer distal to set first air laid paper layer;
a second air laid paper layer distal to send polymer layer;
a polytheylene bottom layer.

In one embodiment the polymer layer distal to set first air laid paper layer is sodium polyacrylate.

In one embodiment the graphene layer is a fabric having graphene disposed therein.

In one embodiment the graphene layer is a fabric having graphene infused within fibers of said fabric.

In one embodiment the graphene layer includes graphene chips.

In one embodiment the proximal non-woven top sheet includes a pair of tabs integral there with an positioned opposite one another along a longitudinal edge.

In one embodiment the polyethylene bottom layer includes a pair of tabs integral there with an positioned opposite one another along a longitudinal edge.

In one embodiment the invention is a hygiene pad consisting of:
a proximal non-woven top sheet;
a graphene layer distal to said nonwoven top sheet;
a first air laid paper layer distal to said graphene layer;
a polymer layer distal to set first air laid paper layer;
a second air laid paper layer distal to send polymer layer;
a polytheylene bottom layer.

The invention in one embodiment, the invention will consist of any combination of the combined components as disclosed herein.

### BRIEF DESCRIPTION OF THE SEVERAL VIEWS OF THE DRAWINGS

FIG. 1 is a perspective separated view of the components according to one embodiment of the present invention.

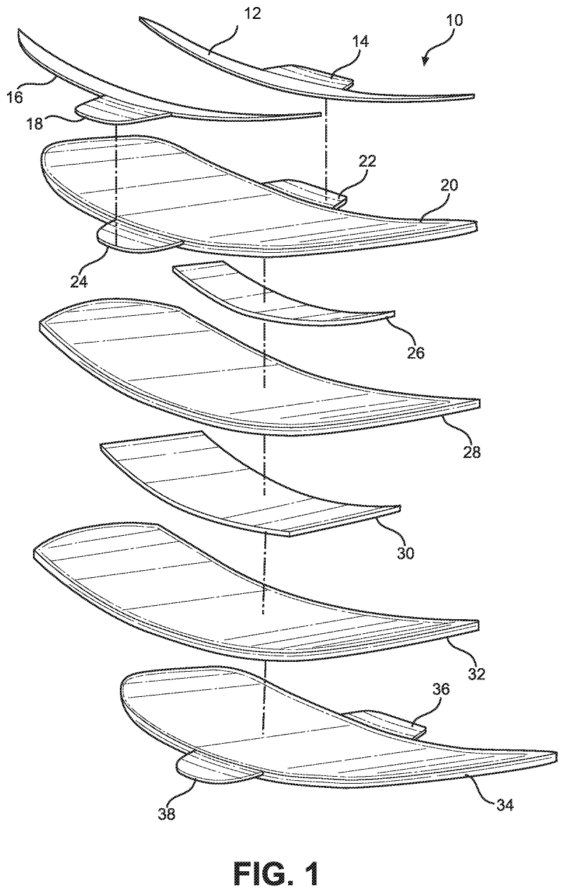

FIG. 2 is a perspective separated view of the components according to one embodiment of the present invention.

### DETAILED DESCRIPTION OF THE PREFERRED EMBODIMENT

In one embodiment of the present invention system 10 is made up of a plurality of components as demonstrated in the drawings and disclosed herein.

The components will be described in terms of proximal being closer to the body and distal being further from the body.

Proximal to the body are first upper leak guard 12 having first upper wing 14 integral there with. Second upper leak guard 16 having second upper wing 18 integral there with. Non-woven top sheet 20 has first lower wing 22 and second lower wing 24 on opposite sides length wide and extending from the longitudinal edge of non-woven top sheet 20. Each of first upper wing 14 second upper wing 18 first lower wing 22 and second lower wing 24 act in concert to inhibit and prevent the pad system from shifting and allows a firm hold to the undergarment.

Distal to nonwoven top sheet 20 is graphene layer 26. As generally understood, this is not pure graphene but rather a breathable fabric material having graphing infused and or disposed there on. In one embodiment, the graphene is present as graphene chips, being separate discrete graphene particles. In one embodiment, non-woven top sheet 20 is a cotton blend.

Further distal to graphene layer 26 is a first air laid paper layer 28. Positioned distal to first air laid paper layer 28 is polymer layer 30. In one embodiment layer 30 is a fabric having sodium polyacrylate disposed thereon or incorporated there with.

Positioned distal to Palmer layer 30 is second air laid paper layer 32. In one embodiment, the final layer is a breathable polyethylene bottom layer 34 having a first side tab 36 and second side tab 38 integral there with.

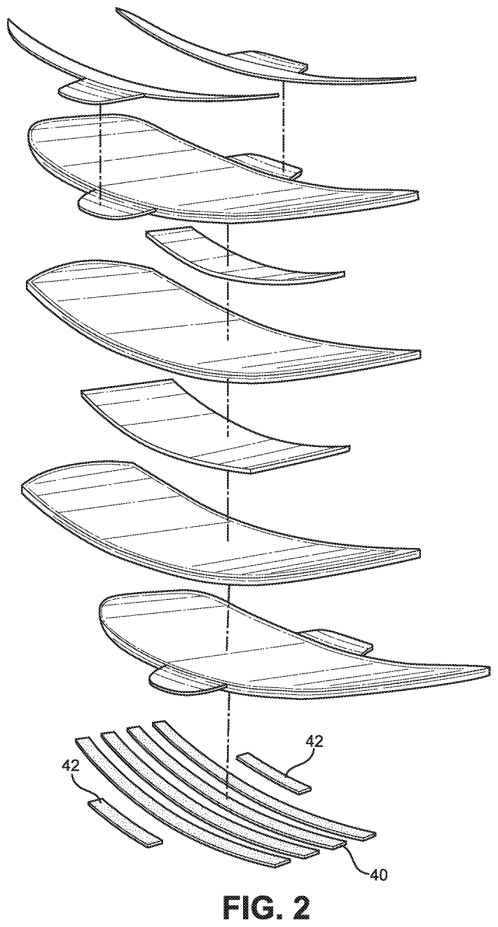

In one embodiment, as demonstrated in FIG. 2, additional structures are positioned distal to polyethylene bottom layer 34 these structures being primary adhesive strips 40 and side adhesive strips 42. The strips are constructed and arranged to have adhesive apply to the undergarment, so the pad doesn't contact the user's skin.

In one embodiment, the invention is formed with a non-toxic polymer has a much better absorbency than traditional sanitary napkins.

As used herein, the Graphene infused strip helps with the pain and aroma.

All layers of the product are porous for breathability which prevents bacteria, heat and moisture sitting against the female's body.

In one embodiment, the polymer is Sodium Polyacrylate.

Graphene has a nano-composite antibacterial factor which eliminates bacteria atoms which contributes to the smell, the lack of chemicals in our pads help with potential pain reduction.

In one embodiment, as demonstrated in the figures, the present invention is constructed as follows:

In one embodiment, the invention includes:

Five successive layers including

5 Layer 5—is SAP′ Super Absorbent Polymer—This layer absorbs the feminine flow up to 10× more than traditional pads.

4 Layer 4—Serves as a Blockade against the moisture.

3 Layer 3—Is infused with Graphene which offers a number of health benefits

2 Layer 2—Top sheet

1 Layer 1—3D extended with wings this prevents leakage onto the under garments.

The unique configuration fulfills the needs that are currently unmet for these products.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A hygiene pad comprising:
a proximal non-woven top sheet;
a graphene layer distal to said nonwoven top sheet;
a first air laid paper layer distal to said graphene layer;
a polymer layer distal to set first air laid paper layer;
a second air laid paper layer distal to send polymer layer;
a polytheylene bottom layer.

2. The hygiene pad of claim 1 wherein said polymer layer distal to set first air laid paper layer is sodium polyacrylate.

3. The hygiene pad of claim 1 wherein said graphene layer is a fabric having graphene disposed therein.

4. The hygiene pad of claim 1 wherein said graphene layer is a fabric having graphene infused within fibers of said fabric.

5. The hygiene pad of claim 1 wherein said graphene layer includes graphene chips.

6. The hygiene pad of claim 1 wherein said proximal non-woven top sheet includes a pair of tabs integral there with an positioned opposite one another along a longitudinal edge.

7. The hygiene pad of claim 1 wherein said polyethylene bottom layer includes a pair of tabs integral there with an positioned opposite one another along a longitudinal edge.
